(19) United States
(12) Patent Application Publication
(10) Pub. No.: US 2011/0196438 A1
(43) Pub. Date: Aug. 11, 2011

(54) THERAPY DEVICE AND METHOD FOR TREATING UNDERLYING TISSUE USING ELECTRICAL AND ACOUSTIC ENERGIES

(76) Inventors: Lukas Mnozil, Litovel (CZ); Tomas Schwarz, Kutna Hora (CZ)

(21) Appl. No.: 13/024,458

(22) Filed: Feb. 10, 2011

Related U.S. Application Data
(60) Provisional application No. 61/303,083, filed on Feb. 10, 2010.

(51) Int. Cl. A61N 7/00; A61N 1/00

(52) U.S. Cl. 607/3

(57) ABSTRACT

A device for subcutaneous adipose tissue skin treatment applies both radio frequency energy (RF) and ultrasound energy (US) into the skin from a single electrode. The electrode may be cooled via a chilled liquid cooling system, thereby indirectly also cooling the skin. An infrared thermometer on the device may measure skin temperature. A passive electrode may be placed on the patient's skin in a location remote from the treatment site.

This Application claims priority to U.S. Provisional Patent Application No. 61/303,083 filed Feb. 10, 2010 and incorporated herein by reference.

### BACKGROUND

Human skin consists of outer epidermal layer, dermal layer and subcutaneous tissue. Various types of energies have been used for treating the skin and subcutaneous tissue structures. The combination is used to increase blood circulation and break adipose tissue cell connections. U.S. Pat. No. 6,325,769 discloses use of ultrasonic (US) energy for skin treatment including applying the acoustic pulse to a subsurface region of the skin without damaging the epidermis layer of the skin. U.S. Pat. No. 6,702,808 discloses using radio frequency (RF) energy to treat complex targets in the skin.

### FIELD OF THE INVENTION

In novel methods and devices, radiofrequency (RF) and ultrasound energy (US) are provided resulting in improved therapeutic effect.

### SUMMARY

An object of the present invention is to provide improved therapeutic method and effect of treating the skin underlying tissue. The present invention is based upon the finding that skin heating improves the blood distribution and US tissue micromassage causes disruption of adipose tissue cells bindings. To obtain these therapeutic effects, both energy types are delivered simultaneously into the skin, without damaging the superfacial layer of the skin.

The superfacial region of the skin to be treated may also be cooled by an RF electrode. Cooling of the epidermal layer creates a reverse thermal gradient where the subcutaneous structures such as adipose tissue is being heated and micromassaged, but the superfacial layer is protected by the continuous cooling.

The RF electrode may optionally be used to monitor skin impedance during treatment. Increasing the skin surface temperature leads to an impedance change. Accordingly, impedance monitoring may be used for optimalization of temperature distribution and RF energy delivery.

In a method of treating the underlying tissue, the temperature of the superfacial skin layer is measured. To control the epidermal layer temperature, an infrared thermometer may be built into the housing of the applicator. The temperature measurement may be provided continuously during the treatment and may also be displayed to the user.

### BRIEF DESCRIPTION OF THE DRAWINGS

FIG. 1 shows a system for treating a skin target and applying RF and US energy into the skin.

FIG. 2 is a bottom perspective view of an applicator device for providing radio frequency and ultrasonic therapy.

FIG. 3 is a top perspective view of the device shown in FIG. 2.

FIG. 4 is a section view of the applicator shown in FIG. 1 and FIG. 2 as fully assembled.

FIG. 5 is a therapy flow chart.

### DETAILED DESCRIPTION OF THE DRAWINGS

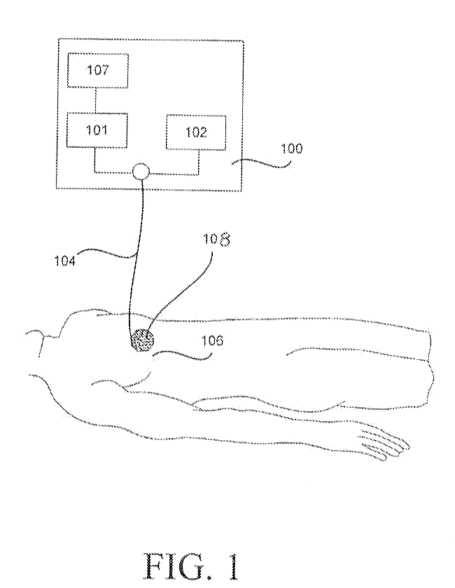

As shown in FIG. 1, a region 108 on the skin 106 of a human patient is identified. An applicator or device 105 is designed to be applied to the region 108 of the skin 106.

The applicator 105 is connected to a control unit 100 via a supply line 104. A pump 101 is used to deliver a cooling fluid to the applicator 105 via a tube in the supply line 104 from a refrigeration unit 107 in the control unit 100. The control unit 100 includes an electrical power supply 102 that is connected to an RF electrode 205 and an US element 311 in the applicator 105 via wires in the supply line 104.

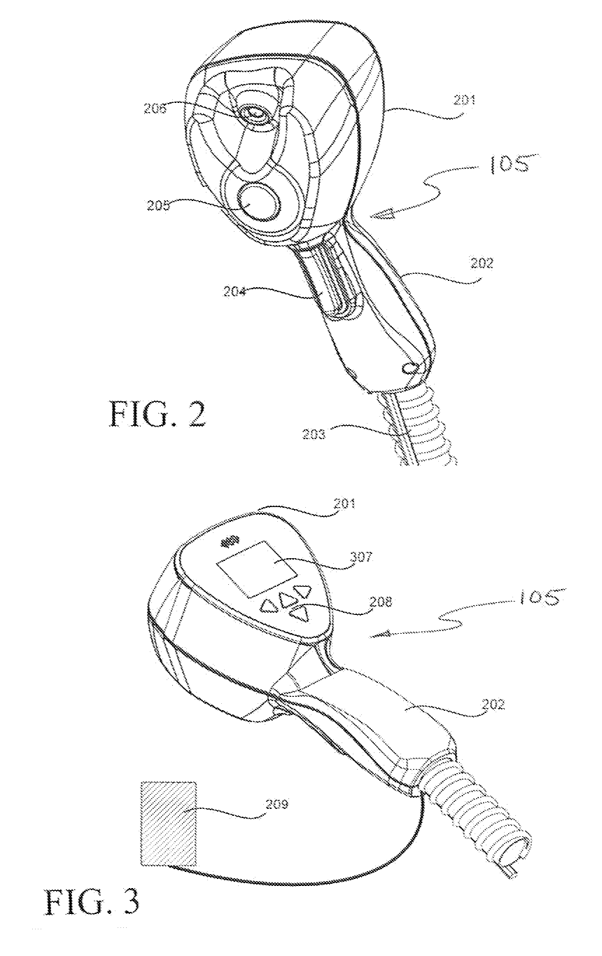

As shown in FIG. 2, the applicator 105 may have a head 201 and a handle 202, with a trigger switch 204 on, the handle 202. The handle 202 is ergonomically designed for grasping in the user's hand. The switch 204 is positioned for actuation by the user's index finger. The RF electrode 205 may be formed in or on part of the head 201. The head 201 may also contain an infrared or other form of thermometer 206, for measuring the skin surface temperature.

As shown in FIG. 3, a digital display 307 and a group of buttons 208 may be provided on the top or back side of the head 201. The buttons 208 may be used to operate device functions and allow the user to input selected values of parameters of the treatment, such as power and intensity. The display 307 may show skin surface temperature.

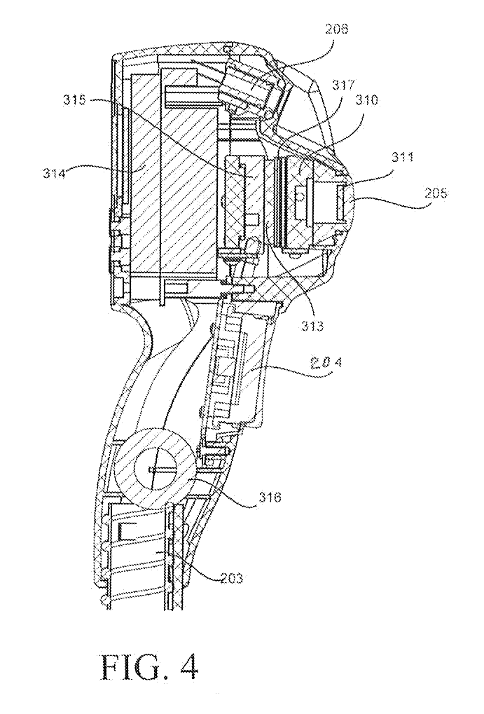

Referring to FIG. 4, the RF electrode 205 may include an US generating piezoelectric element 311. In this case, the RF electrode 205 may have an internal part 310 conductively coupled to an external RF electrode part 205 which projects out from the front or bottom side of the head 201. The internal part of RF electrode 310 is thermally coupled to an isolator, such as a ceramic isolator 317 to conduct heat from the RF electrode 205. The ceramic isolator 317 may be cooled by a Peltier device 313. A maximal temperature of the ceramic isolator may be set to 5° C. A fluid delivery member 315 delivers cooling fluid to the back side of Peltier device 13.

Referring still to FIG. 4, the head 201 includes a control block 314, which provides communication between the applicator 105 and control unit 100. The control block 314 may also obtain information from thermometer 206 measuring the skin surface temperature. The trigger 204 starts and terminates the RF and US energy generation. The handle 202 of the applicator 105 may contain an impedance matching circuit 316 which controlling the impedance between the electrode and the skin.

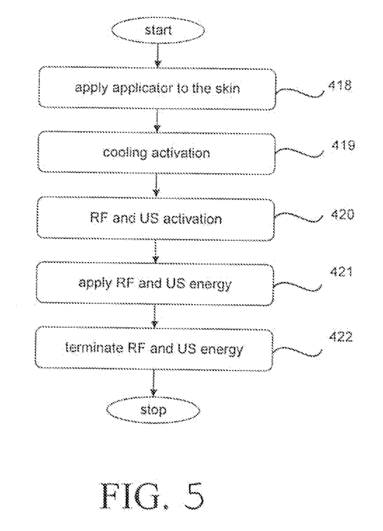

The device 105 may used to perform methods as shown in FIG. 5, wherein RF energy and US energy are simultaneously applied into the skin via the electrode 205. The RF frequency, RF output power, and US power, may be controlled and varied. Referring to FIG. 5, in a first step 418, the applicator 105 is applied to the skin. In step 419 skin cooling is induced by the RF electrode 205. In step 420 the RF and US sources are activated. In step 421 RF and US energy are simultaneously directed into the skin by pressing the trigger button 204. In step 422 the energy emission is terminated by depressing the trigger button. The electrode 205 is cooled by a flow of fluid cooled in the refrigerator unit 103 that flows through fluid delivery member 315.

A passive electrode 209 schematically illustrated in FIG. 3 may be attached to the patient's body opposite the treatment site. An electrically conductive gel may be applied to the patient's skin at the treatment site. The gel may have the acoustic end electric impedance corresponding to the impedance of the treated tissue.

1. A therapy device comprising:
a housing having a head attached to a handle;
a radio frequency energy source and an ultrasonic energy source in the housing;
an electrode on the head of the housing, with the electrode connected to the radio frequency energy source and to the ultrasonic energy source, and with the electrode adapted to provide both radio frequency and ultrasonic stimulation to the skin of a patient; and
a cooling unit associated with the electrode.

2. The device of claim 1 with the cooling unit adapted to cool the electrode and thereby the skin surface.

3. The device according to claim 2 further comprising a refrigeration unit for cooling a fluid, and tubes connecting the refrigeration unit to the cooling unit, to provide a flow of cooling fluid to or adjacent to the electrode.

4. The device according to claim 1 further comprising a thermometer on the housing for measuring a skin surface temperature.

5. The device according to claim 4 wherein the thermometer comprises an infrared thermometer.

6. The device according to claim 4 further comprising an electrical power supply connected to the radio frequency energy source and to the ultrasonic energy source in the housing, and a passive electrode outside Of the housing electrically connected to the power supply.

7. The device according to claim 1 further comprising a digital display and buttons on the housing for selecting treatment parameters and displaying a measured skin surface temperature, respectively.

8. The device according to claim 1 further including a controller for controlling one or more of therapy time, RF frequency, RF energy, RF power, cooling temperature of the fluid, and duty factor of the RF energy.

9. The device according to claim 8 with the controller further controlling US energy, US duty factor and US intensity.

10. The device according to claim 1 wherein the RF energy source provides RF energy at a frequency of from about 300 kHz to about 4 MHz, using an output power from about 1 to about 120 W, and the US energy source provides US energy at a frequency of about 2 MHz and an intensity of from about 0 to about 3 W.

11. A method, comprising:
locating a treatment site on the skin of a human patient;
placing a passive electrode opposite to the treatment site;
applying a conductive gel onto the treatment site;
placing an electrode of an applicator onto the treatment site; and
applying RF energy and US energy simultaneously into the treatment site via the electrode.

12. The method according to claim 11 wherein the energies are applied after pressing the applicator trigger.

13. The method according to claim 11 further comprising cooling the skin by cooling the electrode before, after, or during the therapy.

14. The method according to claim 11 further comprising measuring of skin surface temperature by infrared thermometer

15. The method according to claim 11 wherein output power of the RF energy is from about 1 to about 120 W.

16. The method according to claim 11 wherein output power of the US energy is from about 1 to about 3 W.

17. The method according to claim 11 further comprising deactivating a subcutaneous adipose tissue via the application of the RF and US energy

18. The method of claim 11 further comprising applying the RF and US energy in a time repeating cycle.
